(19) United States
(12) Patent Application Publication
(10) Pub. No.: US 2006/0030603 A1
(43) Pub. Date: Feb. 9, 2006

(54) Novel aryl urea analogs and use thereof as antibacterial agents

(76) Inventors: Punit P. Seth, San Marcos, CA (US); Dale E. Robinson, San Marcos, CA (US); Elizabeth Anne Jefferson, La Jolla, CA (US); Eric E. Swayze, Carlsbad, CA (US)

Correspondence Address: WOODCOCK WASHBURN LLP, ONE LIBERTY PLACE - 46TH FLOOR, PHILADELPHIA, PA, 19103, US

(21) Appl. No.: 10/913,683

(22) Filed: Aug. 5, 2004

(51) Int. Cl. A61K 31/44; A61K 31/17; C07D 213/54; C07C 335/12

(52) U.S. Cl. 514/357; 514/586; 514/595; 546/331; 546/336; 564/27; 564/47

(57) ABSTRACT

Antibacterial compounds are described herein having either formula (I) or formula (II):

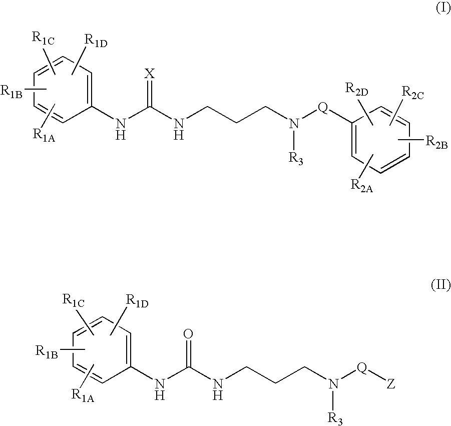

wherein R_{1A}, R_{1B}, R_{1C}, R_{1D}, X, Z, Q, R_{2A}, R_{2B}, R_{2C} and R_{2D} are as defined herein. Compositions comprising compounds of formulas (I) and (II) are also provided.

### FIELD OF THE INVENTION

This invention relates to novel aryl urea analogs and their use, for example, as antibacterial agents. In one aspect, this invention relates to antibacterial compositions comprising novel aryl urea analogs that exhibit low micromolar minimum inhibitory concentrations (MIC) against both Gram-positive and Gram-negative bacteria.

### BACKGROUND OF THE INVENTION

The emergence of drug-resistant, pathogenic bacteria continues to be a serious health problem worldwide. As a result, it has become desirable to identify new structural classes of antibacterial agents to combat the growing threat of bacterial resistance.

A number of recent publications have reported the antibacterial activity of diarylureas derived from aminothiazole, aminopyrazole and haloanilines (see, e.g., Wijkmans, et al., DDT, 2002, 7, 126; Francisco, et al., Med. Chem. Lett. 2004, 14, 235; Kane Jr., et al., Biorg. Med. Chem. Lett. 2003, 13, 4463; Wilson, et al., Biorg. Med. Chem. Lett. 2001, 11, 1149; and Proctor, et al., Antimicrob. Agents Chemother. 2002, 46, 2333). Such diarylureas have been reported to possess excellent activity against Gram-positive bacteria but suffer from poor aqueous solubility and minimal activity in the presence of serum. Accordingly, there exists a need in the art for alternative diarylureas having antibacterial activity.

### SUMMARY OF THE INVENTION

In one aspect, the present invention provides compounds having either formula (I) or formula (II): 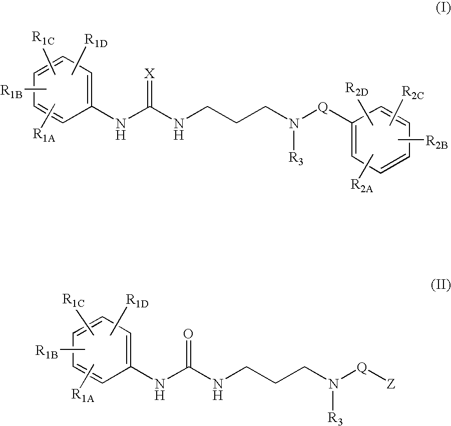
and salts thereof, wherein:

R_{1A}, R_{1B}, R_{1C} and R_{1D} are, independently, H, halogen, C₁-C₃ alkyl, C₁-C₃ haloalkyl, or C₁-C₃ haloalkoxy, provided that at least one of R_{1A}, R_{1B}, R_{1C}, and R_{1D} is not H;

X is O or S;

Q is (CH₂)ₙ or (CH₂)ₙ—O;

n is 1-3;

Z is heteroaryl or substituted heteroaryl;

R_{2A}, R_{2B}, and R_{2C} are, independently, H, halogen, phenyl, C₁-C₃ haloalkoxy, C₁-C₃ alkoxyphenyl, phenoxy, C₁-C₆ alkyl, C₁-C₆ alkoxy, aromatic, heteroaromatic, substituted heteroaromatic, alkylamino, substituted C₁-C₃ haloalkoxy, provided that at least one of R_{2A}, R_{2B}, and R_{2C} is not H; and

R₃ is H or C₁-C₆ alkyl.

Compositions comprising one or more such compounds are also provided, as are methods of using the compounds and compositions for treating a patient suspected of suffering from a disease associated with excessive bacterial activity. Certain methods according to the present invention comprise the step of administering to the patient a therapeutically effective amount of at least one compound of either formula (I) or formula (II).

### DETAILED DESCRIPTION OF ILLUSTRATIVE EMBODIMENTS

The term “alkyl” as used herein, refers to saturated, straight- or branched-chain hydrocarbons having either 1-3 or 1-6 carbon atoms. Examples of alkyl groups include methyl, ethyl, propyl, isopropyl, n-butyl, tert-butyl, neopentyl and n-hexyl groups. The terms “halo” and “halogen,” as used herein, refer to an atom selected from fluorine, chlorine, bromine and iodine.

The term “haloalkyl” denotes an alkyl group, as defined above, having one, or more halogen atoms attached thereto, and is exemplified by such groups as chloromethyl, bromoethyl, trifluoromethyl, and the like. “Haloalkoxy,” in turn, refers to groups having the formula —O-haloalkyl.

The term “aryl,” as used herein, refers to a mono- or bicyclic carbocyclic ring system having one or two aromatic rings including, but not limited to, phenyl, naphthyl, tetrahydronaphthyl, indanyl, idenyl and the like. Preferred aryl groups have up to 10 carbon atoms, preferably up to six carbon atoms. Aryl groups (including bicyclic aryl groups) can be unsubstituted or substituted with one, two or three substituents such as, for example, alkyl, alkyl, haloalkyl, alkoxy, thioalkoxy, amino, alkylamino, dialkylamino, acylamino, cyano, hydroxy, halo, mercapto, nitro, carboxaldehyde, carboxy, alkoxycarbonyl and carboxamide. In addition, the term aryl includes tetrafluorophenyl and pentafluorophenyl groups.

The terms “heteroaryl” or “heteroaromatic,” as used herein, refer to cyclic aromatic groups having from five to ten ring atoms of which at least one one ring atom is selected from S, O and N and the remaining ring atoms are carbon, the radical being joined to the rest of the molecule via any of the ring atoms. Exemplary heteroaryl groups include pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, and isoquinolinyl groups. Heteroaryl groups according to the present invention can bear one or more substituents selected, for example, from halogen, C₁-C₃ alkyl, C₁-C₃ haloalkyl, C₁-C₃ thioalkyl, cyano, nitro, or C₁-C₃ haloalkoxy.

The term “alkoxy” as used herein refers to —O-alkyl groups, wherein “alkyl” is as defined above. Representative alkoxy groups include, for example, methoxy, ethoxy, benzyloxy, t-butoxy, etc.

The term “thioalkyl” as used herein refers to —S-alkyl groups, wherein “alkyl” is as defined above.

The term “aryloxy” as used herein refers to —O-aryl groups, wherein “aryl” is as as defined above. Representative aryloxy groups include, for example, phenoxy and naphthyloxy groups.

The terms “haloalkyl” and “haloalkoxy” as used herein means alkyl, alkenyl or alkoxy, as the case may be, substituted with one or more halogen atoms.

Preferred compounds according to formula (I) are those in which R_{1A}, R_{1B}, R_{1C} and R_{1D} are, independently, H, halogen, C₁-C₃ alkyl, C₁-C₃ haloalkyl, or C₁-C₃ haloalkoxy, provided that at least one of R_{1A}, R_{1B}, R_{1C} and R_{1D} is not H; and R_{2A}, R_{2B}, R_{2C} and R_{2D} are, independently, H, halogen, phenyl, C₁-C₃ haloalkoxy, C₁-C₃ alkoxyphenyl, phenoxy, C₁-C₆ alkyl, C₁-C₆ alkoxy, provided that at least one of R_{2A}, R_{2B}, R_{2C} and R_{2D} is not H.

Preferred compounds according to formula (II) are those in which R_{1A}, R_{1B}, R_{1C} and R_{1D} are, independently, H, halogen, C₁-C₃ alkyl, C₁-C₃ haloalkyl, or C₁-C₃ haloalkoxy, provided that at least one of R_{1A}, R_{1B}, R_{1C} and R_{1D} is not H; and Z is

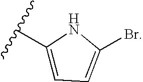

In preferred embodiments of the invention, at least one of R_{1A}, R_{1B}, R_{1C} and R_{1D} is halogen, CF₃, CH₃, or OCF₃ and at least one of R_{2A}, R_{2B}, R_{2C} and R_{2D} is halogen, phenyl, OCF₃, methoxy, ethoxy, phenoxy, or t-Bu. In particularly preferred embodiments, two or more of R_{1A}, R_{1B}, R_{1C} and R_{1D} and two or more of R_{2A}, R_{2B}, R_{2C} and R_{2D} are halogen.

The compounds of formula (I) or formula (II) typically form acid addition salts with organic and inorganic acids. Examples of acid addition salts of compounds of formula (I) or formula (II) are salts with mineral acids, for example hydrohalic acids such as hydrochloric acid, hydrobromic acid and hydriodic acid, sulphuric acid, nitric acid, phosphoric acid and the like, salts with organic sulfonic acids, for example with alkyl- and arylsulfonic acids such as methanesulfonic acid, p-toluene sulfonic acid, benzenesulfonic acid and the like as well as salts with organic carboxylic acids, for example with acetic acid, tartaric acid, maleic acid, citric acid, benzoic acid, salicylic acid, ascorbic acid and the like.

The compounds of formula (I) and formula (II) and their salts can be synthesized by a variety of techniques known to those skilled in the art. One representative synthesis for compounds according to formula (I) is shown in Scheme 1, in which mono-Cbz (benzyloxycarbonyl) protected 1,3 diaminopropane was reacted with 2-trimethylsilylethyl N-succinimidyl carbonate (Teoc-OSu), followed by removal of the Cbz group by catalytic hydrogenation to provide mono-Teoc protected 1,3-diaminopropane 6. Reductive amination of 6 with 3,5-dibromobenzaldehyde, protection of the secondary amine with tert-butoxylcarbonyl anhydride (Boc₂O) and deprotection of the Teoc group using TBAF/KF provided amine 7. Subsequent reaction of 7 with commercially available arylisothiocyanates or arylisocyanates followed by deprotection of the Boc group provided the corresponding thiourea 8 or urea analogs 9 respectively in good overall yield following purification by reversed phase HPLC (60-70%). 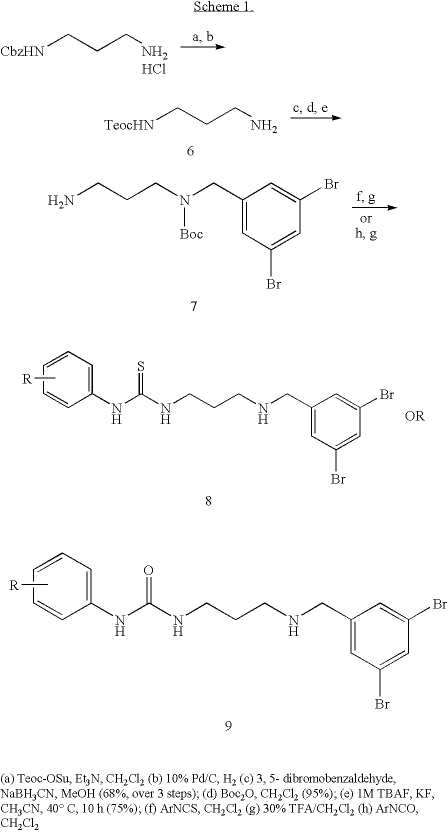
More specifically, commercially available mono-Cbz (benzyloxycarbonyl) protected 1,3-diaminopropane (1 g, 4.08 mmol) was suspended in dry dichloromethane (10 mL). The reaction was cooled in an ice bath and sequentially treated with triethylamine (4.2 mmol, 0.58 mL) and 2-trimethylsilylethyl-N-succinimidyl carbonate (Teoc-Osu, 1.06 g, 4.1 mmol). After stirring at rt for 16 hr, the reaction was diluted with dichloromethane and extracted with 5% HCl, saturated NaHCO₃ solution, brine, dried (MgSO₄) and concentrated. The crude residue obtained was dissolved in ethyl acetate (30 mL) and hydrogenated using 10% Palladium/carbon using a hydrogen balloon. After 16 hr at room temp, the reaction was filtered through celite and the filter bed washed with additional ethyl acetate. The filtrate was concentrated and the crude residue was dissolved in a mixture of dichloromethane (10 mL) and methanol (30 mL). 3,5-Dibromobenzaldehyde (1.05 g, 4 mmol) and glacial acetic acid (1 mL) were added to the reaction. After stirring for 15 minutes at room temp, sodium cyanoborohydride (0.38 g, 6 mmol) was added to the reaction, which was stirred for an additional 16 h at room temp. The reaction was diluted with additional dichloromethane and the organic phase was sequentially washed with sat. NaHCO₃ solution, brine, dried (MgSO₄) and concentrated. The crude residue obtained was purified by chromatography on silica gel using ethyl acetate/hexanes. ¹H NMR (300 MHz, CDCl₃) δ 7.51 (s, 1H), 7.38 (s, 2H), 5.16 (s, br, 1H), 4.12 (t, 2H), 3.69 (s, 2H), 3.25 (m, 2H), 2.64 (t, 2H), 1.65 (m, 2H), 0.95 (t, 2H).

The product (1.26 g) obtained after purification was dissolved in dry dichloromethane and the reaction was treated with tert-butoxycarbonyl anhydride (Boc₂O). After stirring at room temp for 16 hr, the solvent was removed under vacuum and the residue was dissolved in dry acetonitrile (3 mL) and further treated with tetrabutylammonium fluoride (1M solution in THF, 6.9 mL) and potassium fluoride (0.54 g, 9.36 mmol). The reaction was heated at 45 C for 10 hr after which it was diluted with dichloromethane and the organic phase was extracted with water, brine, dried (MgSO₄) and concentrated. The residue was purified by chromatography on silica gel using 1% triethylamine/10-15% methanol/chloroform as the eluant to provide amine 7: ¹H NMR (300 MHz, CDCl₃) δ 7.56 (s, 1H), 7.30 (s, 2H), 4.33 (s, br, 2H), 3.33 (s, br, 2H), 2.76 (s, br, 2H), 1.69 (s, br, 2H), 1.45 (2, br, 9H).

Amine 7 (50 mg, 0.118 mmol) was dissolved in dry dichloromethane (1 mL) and the reaction was treated either with an arylisothiocyanate (0.14 mmol) or an arylisocyanate (0.14 mmol) to provide the corresponding thiourea 8 or the urea 9 respectively, after deprotection of the Boc group using trifluoroacetic acid (0.5 mL). The residue obtained was purified by reversed phase preparative HPLC using a Phenomenex Luna C18 (250×21.2 mm) column, flow rate 30 mL/min, gradient 15-40% buffer B (buffer A—1% glacial acetic acid, pH=3; buffer B—acetonitrile) to provide thioureas 8 or ureas 9.

1-[3-(3,5-Dibromo-benzylamino)-propyl]-3-(3,4-dichloro-phenyl)-thiourea (8k) was prepared according to this general procedure using 3,4-dichlorophenylisothiocyanate to provide thiourea 8k (13 mg as acetate salt). LCMS: M+H=527.8, retention time=2.96 min.

1-[3-(3,5-Dibromo-benzylamino)-propyl]-3-(4-trifluoromethyl-phenyl)-urea (9d) was prepared according to the general procedure using 3-trifluoromethylphenylisocyanate to provide urea 9f (44.5 mg as acetate salt). LCMS: M+H=509.9, retention time=2.93 min.

1-[3-(3,5-Dibromo-benzylamino)-propyl]-3-(3,4-dichloro-phenyl)-urea (9f) was prepared according to the general procedure using 3,4-dichlorophenylisocyanate to provide urea 9c (30.4 mg as acetate salt). LCMS: M+H=509.8, retention time=2.98 min.

Thiourea 8 and urea 9 analogs were tested for antibacterial activity as their acetate salts.

Compounds according to the present invention can also be prepared according to Scheme 2, in which commercially available N-Boc-1,3-diaminopropane was reacted with 4-CF₃-phenylisocyanate followed by deprotection of the Boc group to provide urea 11. Reductive amination with about 40 aromatic aldehydes provides ureas 12 and 13. All the final compounds can be purified by reversed phase HPLC and tested for antibacterial activity as their acetate salts.

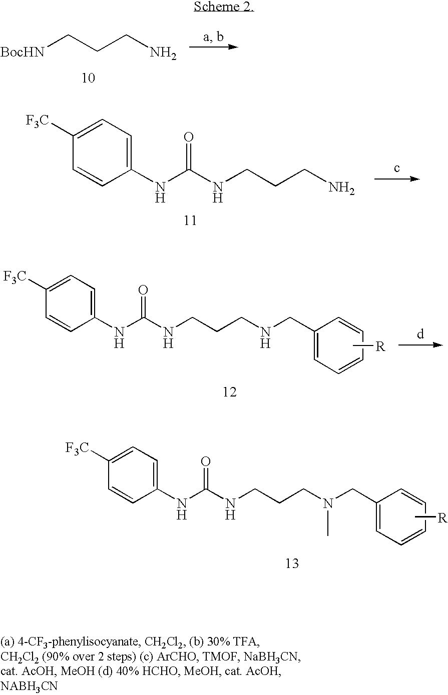

Commercially available amine 10 (0.24 g, 1.37 mmol) was dissolved in dry dichloromethane (7 mL) and the reaction was treated with 4-trifluoromethylphenylisocyanate (0.2 mL, 1.42 mmol). After stirring for 16 hr at room temp, the reaction was concentrated to provide a white solid which was further treated with 50% trifluoroacetic acid in dichloromethane. After stirring for 16 hr at room temp, the reaction was concentrated under vacuum to provide amine 11 (trifluoroacetate salt) as an oil. The oil was then dissolved in dichloromethane and the organic layer was sequentially washed with 4M aqueous sodium hydroxide, brine, dried (MgSO₄) and concentrated to provide amine 11 that was used without any further purification. 11: ¹H NMR (300 MHz, DMSO-d6) δ 8.91 (s, 1H), 7.57 (m, 4H), 6.32 (s, 1H), 3.16 (q, 2H), 2.57 (t, 2H), 1.50 (q, 2H), 1.47 (s, br, 2H).

Amine 11 (0.25 mmol) was dissolved in dry methanol (1 mL), trimethylorthoformate (0.5 mL) and glacial acetic acid (0.031 mL). After stirring for 20 minutes at room temp, the requisite aldehyde (0.25 mmol) dissolved in dichloromethane (0.5 mL) was added and the reaction was stirred for 30 minutes at room temp. Sodium cyanoborohydride (40 mg, 0.3 mmol) dissolved in methanol (0.4 mL) was added to the reaction, which was further stirred at room temp for 16 hr. The reaction was diluted with dichloromethane and washed with sat. NaHCO₃ solution, brine, dried (MgSO₄) and concentrated. The residue obtained was purified by reversed phase preparative HPLC using conditions described in example 1 to provide ureas 12.

1-{3-[(Biphenyl-4-ylmethyl)-amino]-propyl}-3-(4-trifluoromethyl-phenyl)-urea (12e) was prepared according to this general procedure using 4-phenylbenzaldehyde to provide thiourea 12e (55.7 mg as acetate salt). LCMS: M+H=428.1, retention time=2.91 min.

1-[3-(4-Phenoxy-benzylamino)-propyl]-3-(4-trifluoromethyl-phenyl)-urea (12h) was prepared according to the general procedure using 4-phenoxybenzaldehyde to provide thiourea 12e (40.7 mg as acetate salt). LCMS: M+H=444.1, retention time=2.94 min.

1-[3-(4-tert-Butyl-benzylamino)-propyl]-3-(4-trifluoromethyl-phenyl)-urea (12i) was prepared according to the general procedure using 4-tertbutylbenzaldehyde to provide thiourea 12i (61.9 mg as acetate salt). LCMS: M+H=408.1, retention time=2.96 min.

1-[3-(3,5-Dibromo-2-ethoxy-benzylamino)-propyl]-3-(4-trifluoromethyl-phenyl)-urea (12s) was prepared according to the general procedure using 4-tertbutylbenzaldehyde to provide thiourea 12s (59.9 mg as acetate salt).

Compounds of the present invention can also be prepared according to Scheme 3, in which replacement of the secondary amine in the tether with a tertiary amine was effected. 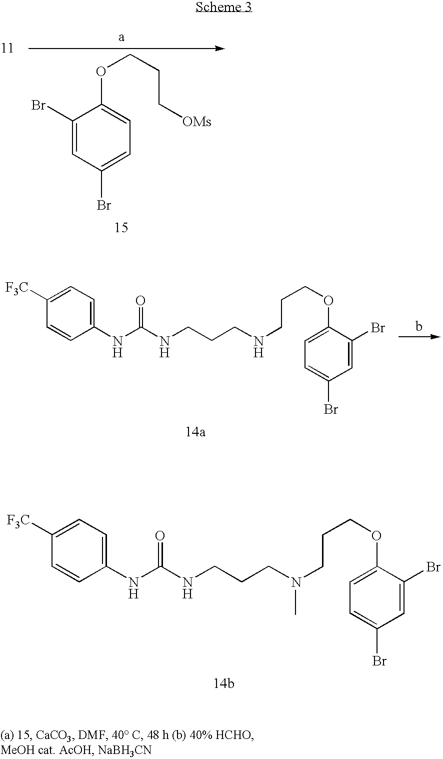
To prepare mesylate 15, a mixture of commercially available 2,4-dibromophenol (0.5 g, 1.98 mmol), 3-bromopropanol (0.164 mL, 1.88 mmol) and potassium carbonate (0.27 g, 2 mmol) in acetone (6 mL) was stirred at 55 C for 16 hr. The reaction was diluted with ethyl acetate and the organic phase was sequentially washed with sat. NaHCO₃ solution, water, brine, dried (MgSO₄) and concentrated. The crude alcohol thus obtained was dissolved in dry dichloromethane (10 mL) and the reaction was cooled in an ice bath. Dimethylaminopyridine (catalytic), triethylamine (0.3 mL, 2.5 mmol) were added to the reaction followed by drop-wise addition of methanesulfonyl chloride (0.16 mL, 2 mmol). After stirring for 16 hr at room temp, the reaction was diluted with dichloromethane and the organic layer was sequentially washed with 5% HCl, sat. NaHCO₃ solution, brine, dried (MgSO₄) and concentrated to provide crude mesylate 15, which was used without any further purification. 15: ¹H NMR (300 MHz, CDCl₃) δ 7.66 (d, 1H), 7.38 (dd, 1H), 6.76 (d, 1H), 4.50 (t, 2H), 4.13 (t, 2H), 3.00 (s, 3H), 2.30 (m, 2H).

A mixture of amine 11 (0.92 g, 3.34 mmol) prepared generally according to Scheme 2, crude mesylate 15 obtained above and CsCO₃ (1.3 g, 4 mmol) in dimethylformamide (4 mL). was stirred at 40 C for 48 hr. The reaction was then diluted with dichloromethane and filtered through celite and the filtrate was concentrated to provide crude 14a. Some of the crude residue was purified by reversed phase preparative HPLC to provide pure urea 14a for biological screening. The rest of the crude residue was dissolved in a mixture of methanol (10 mL), formaldehyde (30% aqueous solution, 1.5 mL) and glacial acetic acid (10 drops) and then treated with Sodium cyanoborohydride (0.37 g, 10 mmol). After strring at room temp for 16 hr, the reaction was diluted with dichloromethane and the organic phase was washed with sat. NaHCO₃ solution, brine, dried (MgSO₄) and concentrated. The crude residue was purified by chromatography on silica gel (1% ammonium hydroxide/10% methanol/chloroform) to provide 14b (1 g). The pure urea 14b was dissolved in dry 1,4-dioxane (5 mL) and further treated with hydrochloric acid (4M solution in 1,4-dioxane, 8.8 mmol, 2.2 mL). The HCl salt of urea 14b (1.07 g) that precipitates out was collected by filtration and dried under high vacuum.

1- {3-[3-(2,4-Dibromo-phenoxy)-propylamino]-propyl}-3-(4-trifluoromethyl-phenyl)-urea (14a). Obtain 130.6 mg as acetate salt after preparative HPLC purification. LCMS: M+H=553.9, retention time=3.02 min.

1-(3-{[3-(2,4-Dibromo-phenoxy)-propyl]-methyl-amino}-propyl)-3-(4-trifluoromethyl-phenyl)-urea. (14b). ¹H NMR (300 MHz, CDCl₃) δ 7.66 (d, 1H), 7.48 (d, 2H), 7.43 (d, 2H), 7.31 (dd, 1H), 6.71 (d, 1H), 4.06 (t, 2H), 3.35 (m, 2H), 2.65 (t, 2H), 2.56 (t, 2H), 2.29 (s, 3H), 1.98 (m, 2H), 1.74 (m, 2H). LCMS: M+H=567.9, retention time=3.01 min.

Compounds according to the present invention can also be prepared according to Scheme 4. 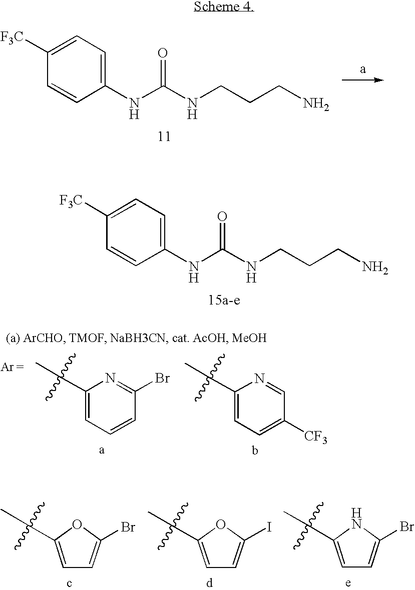
Compounds 15a-e were prepared according to the general procedure of Scheme 2 using the appropriate substituted heteroaryl aldehydes and amine 11. For example, 1-{3-[(5-bromo-1H-pyrrol-2-ylmethyl)-amino]-propyl}-3-(4-trifluoromethyl-phenyl)-urea (15e) was prepared according to this procedure using 2-bromo-pyrrole-4-carboxaldehyde to provide thiourea 15e (16.6 mg as acetate salt). LCMS: retention time=2.99 min.

In vitro structure/activity relationship (“SAR”) tests were performed according to the following procedure: MIC assays were carried out in a 150 μL volume in duplicate in 96-well clear flat-bottom plates. The bacterial suspension from an overnight culture growth in the appropriate medium was added to a solution of test compound in 0.5% DMSO in water. Final bacterial inoculum was approximate 10³-10⁴ CFU/well. The percentage growth of the bacteria in the test wells relative to that observed for a control well containing no compound was determined by measuring absorbance at 595 nm (A₅₉₅) after 20-24 h at 37° C. The MIC was determined as a range of concentrations where complete inhibition of growth was observed at the higher concentration and the bacterial cells were viable at the lower concentration. The bacterial strains used for the assays include S. aureus ATCC 13709, S. pyogenes ATCC 49399, E. faecalis ATCC 29212, E. faecium ATCC 6569, E. coli ATCC 25922, K. pneumoniae ATCC 13383, P. vulgaris ATCC 8427, P. aeruginosa ATCC 25416. The results of the in vitro SAR tests are in Tables 1-3.

TABLE 1
In vitro antibacterial activity of thiourea/urea analogs

| | | | MIC (μM) |
|---|---|---|---|
| Entry | Compd | R | S. aureus |
| 1 | 8a | 2-F | 25-50 |
| 2 | 8b | 2-Cl | 12-25 |
| 3 | 8c | 2-OMe | >100 |
| 4 | 8d | 2-SMe | 50-100 |
| 5 | 8e | 2,3,4-triF | 6-12 |
| 6 | 8f | 3-F | 12-25 |
| 7 | 8g | 3-Cl | 6-12 |
| 8 | 8h | 3-Br | 6-12 |
| 9 | 8i | 3-CF₃ | 6-12 |
| 10 | 8j | 3-OMe | 25-50 |
| 11 | 8k | 3,4-diCl | 3-6 |
| 12 | 8l | 4-F | 12-25 |
| 13 | 8m | 4-Cl | 6-12 |
| 14 | 8n | 4-Br | 12-25 |
| 15 | 8o | 4-I | 12-25 |
| 16 | 8p | 4-CF₃ | 6-12 |
| 17 | 8q | 4-NO₂ | 12-25 |
| 18 | 8r | 4-CH₃ | 12-25 |
| 19 | 8s | 4-CN | 25-50 |
| 20 | 8t | 4-OMe | 25-50 |
| 21 | 8b | 2-SMe | 25-50 |
| 22 | 9a | 2-OMe | 25-50 |
| 23 | 9b | 3-CF₃ | 6-12 |
| 24 | 9c | 4-Cl | 6-12 |
| 25 | 9d | 4-CF₃ | 3-6 |
| 26 | 9e | 4-SMe | 25-50 |
| 27 | 9f | 3,4-diCl | 3-6 |
| 28 | 9g | 4-OCF₃ | 6-12 |
| 29 | Linezolide | — | 3-6 |

TABLE 2
In vitro antibacterial activity of benzylamino group modified analogs.

| | | | MIC (μM) |
|---|---|---|---|
| Entry | Compd | R | S. aureus |
| 1 | 12a | H | 50-100 |
| 2 | 12b | 2-OCF₃ | 12-25 |
| 3 | 12c | 3-Ph | 6-12 |
| 4 | 12d | 4-OCF₃ | 3-6 |
| 5 | 12e | 4-Ph | 3-6 |
| 6 | 12f | 4-(4′-OMe)-Ph | 3-6 |
| 7 | 12g | 4-(2′-OMe)-Ph | 6-12 |
| 8 | 12h | 4-OPh | 3-6 |
| 9 | 12i | 4-t-Bu | 3-6 |
| 10 | 12j | 4-NMe₂ | 12-25 |
| 11 | 12k | 4-NHCOMe | >100 |
| 12 | 12l | 3,5-diBr | 3-6 |
| 13 | 12m | 3,5-diCl | 6-12 |
| 14 | 12n | 3,5-diF | 50-100 |
| 15 | 12o | 3,5-diCF3 | 50-100 |
| 16 | 12p | 3,5-diOMe | 50-100 |
| 17 | 12q | 3,4-diCl | 3-6 |
| 18 | 12r | 2-OH-3,5-diBr | >100 |
| 19 | 12s | 2-OEt-3,5-diBr | 3-6 |
| 20 | 12t | 2-OH-3-OMe-5-Br | 12-25 |
| 21 | 12u | 2-OEt-5-Br | 6-12 |
| 22 | 13 | 3,5-diBr | >100 |
| 23 | 14a | — | 3-6 |
| 24 | 14b | — | 3-6 |
| 23 | Linezolide | — | 3-6 |

TABLE 3
In vitro antibacterial activity of heteroaryl analogs.

| | | MIC (μM) |
|---|---|---|
| Entry | Compd | S. aureus |
| 1 | 15a | 50-100 |
| 2 | 15b | 50-100 |
| 3 | 15c | 50-100 |
| 4 | 15d | 50-100 |
| 5 | 15e | 3-6 |

A number of analogs were also evaluated against a broader panel of Gram-positive and Gram-negative bacteria, as shown in Table 4.

TABLE 4
Broad spectrum activity of selected urea analogs

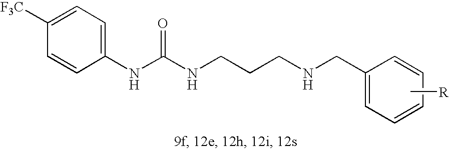

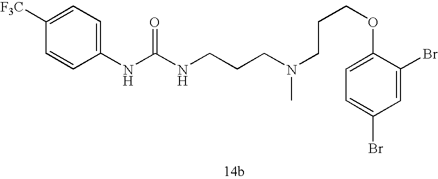

| | MIC | | | | | | |
|---|---|---|---|---|---|---|---|
| | (μM) | | | | | | |
| | | 12s | | | | | |
| | 9f | 2-OEt-3,5- | 12e | 12h | 12i | | |
| Bacteria | 3,5diBr | diBr | 4-Ph | 4-OPh | 4-t-Bu | 14b | Linezolide |
| S. aureus ATCC 13709 | 3-6 | 3-6 | 3-6 | 3-6 | 3-6 | 3-6 | 3-6 |
| S. pyogenes ATCC 49399 | 6-12 | 3-6 | 3-6 | 3-6 | 3-6 | 3-6 | 1.5-3 |
| E. faecalis ATCC 29212 | 6-12 | 6-12 | >100 | 3-6 | 6-12 | 3-6 | 3-6 |
| E. faecium ATCC 6569 | >100 | 6-12 | >100 | >100 | 3-6 | >100 | >100 |
| E. coli ATCC 25922 | 12-25 | 12-25 | 6-12 | 6-12 | 6-12 | 12-25 | >100 |
| K. pneumoniae ATCC | 6-12 | 3-6 | 6-12 | 6-12 | 3-6 | 3-6 | >100 |
| 13383 | | | | | | | |
| P. vulgaris ATCC 8427 | 12-25 | 12-25 | 12-25 | 12-25 | 12-25 | 12-25 | 12-25 |
| P. aeruginosa ATCC 25416 | >100 | >100 | >100 | >100 | >100 | >100 | >100 |
| S. aureus ATCC 13709(+ | 25-50 | 25-50 | 12-25 | 12-25 | 50-100 | 25-50 | 3-6 |
| 4% BSA) | | | | | | | |
| MIT HUH-7-CC₅₀ 48h | NT | 50-100 | 25-50 | 12.5-25 | NT | >100 | NT |
| (μM) | | | | | | | |
| log P | 4.89 | 5.1 | 4.91 | 4.77 | 4.94 | 5.3 | 0.58 |

Analogs 12e and 14b were tested in vivo in a lethal murine model of bacterial infection, as shown in Table 5. The in vivo tests were preformed according to the following procedure:

Mouse Protection Assay: 10 mice/dose group (ICR-CD-1 female mice 18-20 grams, Charles River) were infected with a lethal dose (10⁶ CFU/mouse) of S. aureus (ATCC 13709) suspended in 7.5% hog Gastric Mucin IP. The infected animals were treated at 1 h and 3 h post infection with either compound 12e (lactate salt) from 37 mg/kg down to 2.3 mg/kg or compound 14b (HCl salt) from 75 mg/kg down to 2.3 mg/kg (0.1 mL/mouse). The positive control drug was Vancomycin (Eli Lilly) 1 mg/kg. The animals were observed for one week and mortality was calculated.

Acute Toxicity Study: The maximum tolerated dose for either compound 12e or 14b was determined by administering the compound from 150 mg/kg down to 19 mg/kg given either intraperitoneally (IP) or subcutaneously (subQ). The animals were observed for seven days. In the subQ group all animals survived at all doses tested. On autopsy some of the compound appeared to precipitate at the injection site thereby reducing the effective dose. Via the IP route compound 12e was toxic down to 37 mg/kg and compound 14b was only toxic at 150 mg/kg.

TABLE 5
In vivo antibacterial activity of urea analogs.

| | | Dose | Mice |
|---|---|---|---|
| Entry | Compd | mg/Kg | Alive/Total |
| 1 | No drug | — | 0/10 |
| 2 | 14b | 75 × 2 | 4/10 |
| 3 | 14b | 37.5 × 2 | 7/10 |
| 4 | 14b | 18.8 × 2 | 6/10 |
| 5 | 14b | 9.4 × 2 | 6/10 |
| 6 | 14b | 4.7 × 2 | 4/10 |
| 7 | 14b | 2.3 × 2 | 3/10 |
| 8 | 12e | 37.5 × 2 | 3/10 |
| 9 | 12e | 18.8 × 2 | 3/10 |
| 10 | 12e | 9.4 × 2 | 4/10 |
| 11 | 12e | 4.7 × 2 | 2/10 |
| 12 | 12e | 2.3 × 2 | 0/10 |
| 13 | Vancomycin | 1 × 2 | 10/10 |

Without being bound to any particular theory, it would appear that the methyl group in 13 may alter the orientation of the tether such that it is not able to bind its target in the bioactive conformation. In contrast, the extended tether analogs may be flexible enough to adopt the bioactive conformation despite the methyl group on the tether amine. Analysis of the above data would also suggest that the urea analogs are binding in a very hydrophobic binding pocket. Consequently, increasing hydrophobicity improves the activity of these compounds. The antibacterial activity of the urea analogs also appears to be reduced in the presence of 4% bovine serum albumin. It is conceivable that the reduced activity could be attributed to high serum protein binding of this compound class. These observations are consistent with the previous SAR studies carried out on other urea based antibacterial compounds.

The present invention also includes pharmaceutical compositions and formulations that include the compounds and compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compounds and compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients. One of skill in the art will recognize that formulations are routinely designed according to their intended use, i.e., route of administration. Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Compounds suitable for use in the practice of this invention can be administered orally. The amount of a compound of the present invention in the composition can vary widely depending on the type of composition, size of a unit dosage, kind of excipients, and other factors well known to those of ordinary skill in the art. In general, the final composition can comprise from, for example, 0.000001 percent by weight (% w) to 10% w of the compound, preferably 0.00001% w to 1% w, with the remainder being the excipient or excipients. Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Although the invention has been described with respect to various preferred embodiments, it is not intended to be limited thereto, but rather those skilled in the art will recognize that variations and modifications may be made therein which are within the spirit of the invention and the scope of the appended claims.

What is claimed:

1. A compound having either formula (I) or formula (II): 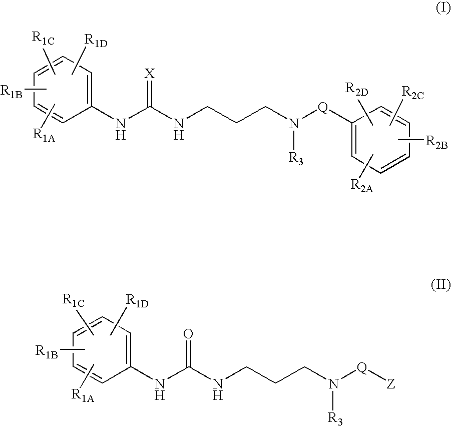
and salts thereof, wherein:
wherein
R_{1A}, R_{1B}, R_{1C} and R_{1D} are, independently, H, halogen, C₁-C₃ alkyl, C₁-C₃ haloalkyl, or C₁-C₃ haloalkoxy, provided that at least one of R_{1A}, R_{1B}, R_{1C}, and R_{1D} is not H;
X is O or S;
Q is (CH₂)ₙ or (CH₂)ₙ—O;
n is 1-3;
Z is heteroaryl or substituted heteroaryl;
R_{2A}, R_{2B}, R_{2C} and R_{2D} are, independently, H, halogen, phenyl, C₁-C₃ haloalkoxy, C₁-C₃ alkoxyphenyl, phenoxy, C₁-C₆ alkyl, C₁-C₆ alkoxy, provided that at least one of R_{2A}, R_{2B}, R_{2C} and R_{2D} is not H; and
R₃ is H or C₁-C₆ alkyl.

2. The compound of claim 1 wherein at least one of R_{1A}, R_{1B}, R_{1C} and R_{1D} is halogen, CF₃, CH₃, or OCF₃.

3. The compound of claim 1 wherein two or more of R_{1A}, R_{1B}, R_{1C} and R_{1D} are halogen.

4. The compound of claim 1 wherein at least one of R_{2A}, R_{2B}, R_{2C} and R_{2D} is halogen, phenyl, OCF₃, methoxy, ethoxy, phenoxy, or t-Bu.

5. The compound of claim 1 wherein two or more of R_{2A}, R_{2B}, R_{2C} and R_{2D} are halogen.

6. The compound of claim 1 wherein Q is CH₂.

7. The compound of claim 1 wherein Q is (CH₂)₃—O.

8. The compound of claim 1 wherein
R_{1A} and R_{1C} are each H;
R_{1B} is 4-CF₃;
R_{2A} and R_{2C} are each H;
R_{2B} is 4-OCF₃, 4-Ph, 4-(4′-OMe)-Ph, 4-OPh, 4-t-Bu, 3,5-di Br, 3,4-di Cl, or 2-OEt-3,5-di Br; and
R₃ is H.

9. The compound of claim 1 wherein Z has structure a, b, c, d, or e:

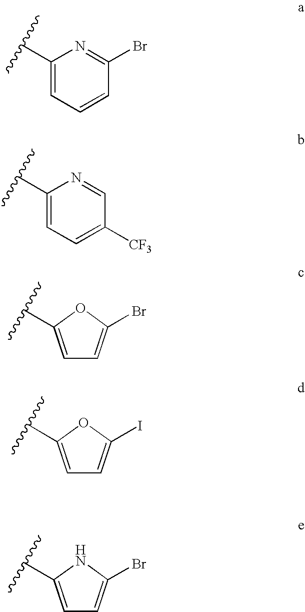

10. A method comprising contacting bacteria with at least one compound according to claim 1.

11. The method of claim 10 wherein the contacting is effected in vitro.

12. The method of claim 10 wherein the contacting is effected in vivo.

13. The method of claim 10 further comprising determining the activity of said bacteria.

14. The method of claim 13 wherein said determination is made before said contacting step.

15. The method of claim 13 wherein said determination is made after said contacting step.

16. A method for treating a patient suspected of suffering from a disease associated with excessive bacterial activity, comprising the step of administering to the patient a therapeutically effective amount of at least one compound of claim 1.

17. A composition comprising at least one compound of claim 1 and a carrier or diluent therefor.

18. A method comprising contacting bacteria with at least one composition according to claim 17.

19. The method of claim 18 wherein the contacting is effected in vitro.

20. The method of claim 18 wherein the contacting is effected in vivo.

21. The method of claim 18 further comprising determining the activity of said bacteria.

22. The method of claim 21 wherein said determination is made before said contacting step.

23. The method of claim 21 wherein said determination is made after said contacting step.
